(19) United States
(12) Patent Application Publication
(10) Pub. No.: US 2025/0020581 A1
(43) Pub. Date: Jan. 16, 2025

(54) Residential Water Quality Monitoring System and Methods of Use Thereof

(71) Applicant: Christopher B. Milligan, Fayetteville, AR (US)

(72) Inventor: Christopher B. Milligan, Fayetteville, AR (US)

(73) Assignee: Christopher B. Milligan, Fayetteville, AR (US)

(21) Appl. No.: 18/773,171

(22) Filed: Jul. 15, 2024

Related U.S. Application Data
(60) Provisional application No. 63/513,780, filed on Jul. 14, 2023.

(51) Int. Cl. G01N 21/31; G01N 33/18

(52) U.S. Cl. CPC G01N 21/31; G01N 33/18

(57) ABSTRACT

Systems for monitoring water quality which are installed in a toilet reservoir and methods of using the same are provided. The system including a housing configured to be at least partially submerged in water in the toilet reservoir, a computing device disposed in the housing and in data communication with a database, and at least one sensor in data communication with the computing device. The at least one sensor measuring at least one water quality parameter. The at least one sensor takes a measurement of the at least one water quality parameter and transmits the measurement to the computing device and the computing device stores the measurement on the database.

### CROSS-REFERENCE TO RELATED APPLICATION

This application claims priority to U.S. Provisional Patent Application Ser. No. 63/513,780, filed Jul. 14, 2023, the contents of which are hereby incorporated by reference in its entirety.

### TECHNICAL FIELD

The present disclosure relates to a residential water quality monitoring system. More specifically, the present disclosure relates to a quick installation, low-cost system that is installed within a toilet reservoir that monitors and records of various water quality parameters in real time.

### BACKGROUND

Water quality is a major concern for homeowners, renters, water utilities, and municipalities alike, with potential risks ranging from poor taste and odor to serious health hazards such as lead contamination. According to the EPA, approximately 15% of Americans are served by public water systems that violate health-based standards, and many more rely on private wells that may be contaminated with harmful pollutants.

Most municipalities and local governments require that annual water tests be conducted and reported to consumers. However, potential hazards and contaminates in the water may be flushed from the system between such infrequent testing, thereby leaving consumers unaware of the hazards or contaminates in their water. Further such reports can be difficult to understand by ordinary consumers, so even when a potential hazard or contaminate is identified, consumers are still unaware.

Additionally, testing is typically carried out at treatment facilities, reservoirs, or other bodies of water that service multiple end users, i.e., water is typically tested at the source and not where end users receive the water. However, testing at the source does not safeguard end users from possible contamination downstream. For example, a cracked or broken pipe, heavy metal leaching, or even intentional/unintentional back contamination by end users.

Current water quality monitoring systems are often expensive to the point where they are cost prohibitive to residential consumers and installing these quality monitoring systems can be complex, requiring the water be shut off and/or disassembling pipes to install. Many residential consumers feel uncomfortable doing such in-depth installation procedures opting to use a plumber, which further adds to the complexity and cost of the monitoring system.

Thus, there exists a need for a water quality monitoring system to provide consumers with a convenient and easy to install monitoring system that is low cost to ensure the safety and quality of their water.

This section is intended to introduce the reader to various aspects of art that may be related to various facets of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

According to an aspect of the present disclosure a system for monitoring water quality installed in a toilet reservoir is provided. The system including a housing configured to be at least partially submerged in water in the toilet reservoir, a computing device disposed in the housing and in data communication with a database, and at least one sensor in data communication with the computing device. The at least one sensor measuring at least one water quality parameter. The at least one sensor takes a measurement of the at least one water quality parameter and transmits the measurement to the computing device and the computing device stores the measurement on the database.

According to another aspect of the preset disclosure a system for monitoring water quality is provided. The system including a server and a plurality of monitoring devices installed in a respective water tank. Each of the monitoring devices including a housing configured to be at least partially submerged in water in the respective water tank, a computing device disposed in the housing and in data communication with the server, and at least one sensor in data communication with the computing device. The at least one sensor measuring at least one water quality parameter. The at least one sensor takes a measurement of the at least one water quality parameter in the respective water tank and transmits the measurement to the computing device and the computing device stores the measurement on the server. The server compiles and transmits the measurement from each of the monitoring device to a user device to be displayed.

According to a further aspect of the present disclosure, a method for using a device for monitoring water quality installed in a toilet reservoir is provided. The method including measuring at least one water quality parameter using one or more sensors. Transmitting and storing the measurements of the at least one water quality parameter on a server. Determining a second water quality parameter from the measurements of the at least one water quality parameter. Transmitting the measurements of the at least one water quality parameter and the second water quality parameter to a user device for display.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is a schematic illustration of an exemplary computing device for use with a water quality monitoring device;

FIG. 2 is a schematic illustration of an exemplary network for use with a water quality monitoring system;

FIG. 3 is a block diagram of water quality monitoring system;

FIG. 4. is a perspective view of a water quality monitoring device which has been constructed in accordance with an embodiment of the present disclosure;

FIG. 5 is a perspective view of the water quality monitoring device of FIG. 4 installed in a water tank;

FIG. 6 is a perspective view of a modular water quality monitoring device; and

FIG. 7 is a flow diagram depicting a method for using a water quality monitoring system.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

### DETAILED DESCRIPTION

The following detailed description and examples set forth preferred materials, components, and procedures used in accordance with the present disclosure. This description and these examples, however, are provided by way of illustration only, and nothing therein shall be deemed to be a limitation upon the overall scope of the present disclosure.

Water quality is a significant concern for homeowners and water utilities. There is a growing need for a cost-effective and reliable system that can monitor water quality in real time and provide useful information to consumers and utilities. The present disclosure addresses this problem by providing a residential water quality monitoring system that is accessible to the average consumer and capable of real time monitoring and recording of multiple water quality parameters.

The present disclosure relates to a residential water quality monitoring system that is installed in a toilet reservoir. The water quality monitoring system can include a multi-parameter probe that is capable of real time monitoring and recording of multiple water quality parameters, including pH, alkalinity, minerals, and other key indicators of water quality. The system is connected to the cloud via a local network, allowing users to access data and insights in real-time through an app-enabled interface. The app displays instantaneous data as well as historical logs and trends and can make recommendations and warnings based on both directly measured and inferred data. The location of the system in a toilet reservoir, allows for easy installation and provides consistent water change out for accurate measurements. The system is designed to be user-friendly, with simple installation and intuitive app interface. It is also flexible, with the ability to incorporate a wide range of water quality instruments and sensors to meet the specific needs of different users. In order to accomplish this the system includes a one or more of modular sensors that can be added, removed, and switched out as desired. Furthermore, a computing system receives various signals gathered by the one or more sensors and sends information to external electronic systems. Thus, the present disclosure addresses the problem of water quality monitoring, providing consumers with a convenient and effective tool for ensuring the safety and quality of their water.

According to some embodiments of the present disclosure, the system may digitally collect and store water quality data, creating a history which allows users to track and analyze the water quality over time. In some embodiments, the historic water quality data can be accessed by a third party, such as a utility or service provider on a computing device, such as a smartphone internet access, and web browser.

In accordance with some embodiments of the present disclosure, the system may be configured with modules able to provide analysis of the water quality data using artificial intelligence or machine learning means, including, but not limited to, machine learning models trained on various amounts of test and training data, neural networks (e.g., Artificial Neural Networks (ANN), Convolution Neural Networks (CNN), Recurrent Neural Networks (RNN)), deep learning models and deep-learning-based generative models (e.g., generative adversarial networks (GANs)). One of ordinary skill in the art would appreciate that there are numerous types of ML and AI systems that could be used for the purposes detailed herein, and embodiments of the present disclosure are contemplated for use with any such ML or AI systems. This may also help utility or service providers to document and track water quality trends to be able to identify water quality metrics that are not explicitly measured. The analysis may also deliver notifications regarding preventive measures to utility or service provides and/or end users.

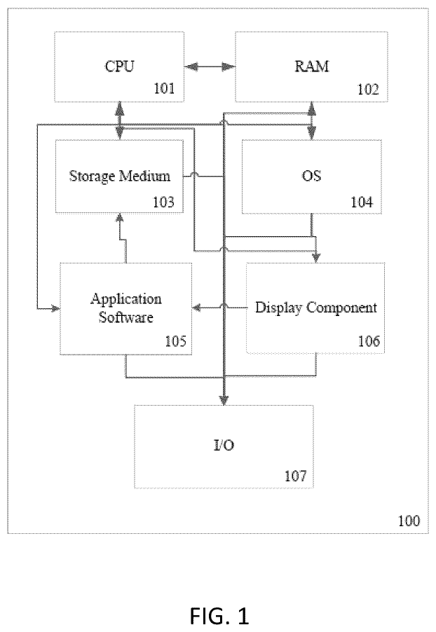

Turning now to FIG. 1, an illustrative representation of a computing device 100 appropriate for use with some embodiments of the system of the present disclosure is shown. The computing device 100 can generally be comprised of a Central Processing Unit (CPU, 101), optional further processing units including a graphics processing unit (GPU), a Random Access Memory (RAM, 102), a mother board 103, or alternatively/additionally a storage medium (e.g., hard disk drive, solid state drive, flash memory, cloud storage), an operating system (OS, 104), one or more application software 105, a display element 106 (e.g., an indicator light, monitor, capacitive touchscreen), and one or more input/output devices/means 107, including one or more communication interfaces (e.g., RS232, Ethernet, WiFi, Bluetooth, USB, wired connection). In some embodiments, the computing device 100 does not include a display component 106 and may transmit information via the input/output device 107 to be displayed on an external device.

According to some embodiments of the present disclosure, data (e.g., water quality data) may be transferred to the system, stored by the system and/or transferred by the system to users of the system across local area networks (LANs) or wide area networks (WANs). In accordance with some embodiments, the system may be comprised of numerous servers, mining hardware, computing devices, or any combination thereof, communicatively connected across one or more LANs and/or WANs. One of ordinary skill in the art would appreciate that there are numerous manners in which the system could be configured and embodiments of the present disclosure are contemplated for use with any configuration.

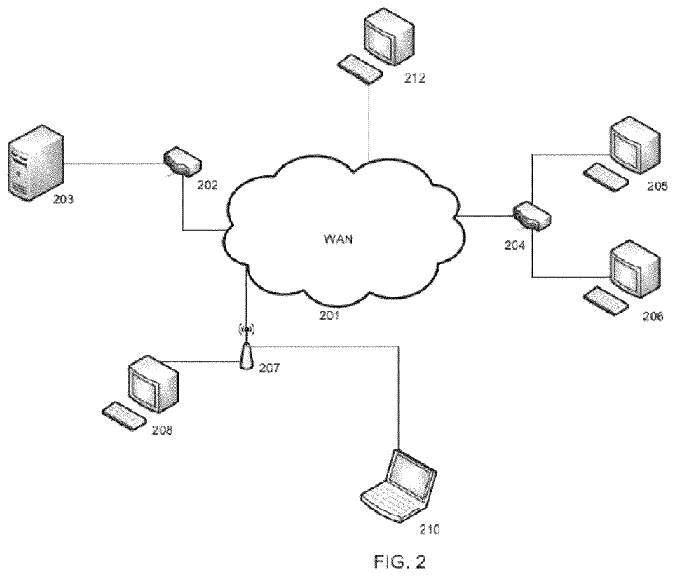

Referring to FIG. 2, a schematic overview of a system in accordance with an embodiment of the present disclosure is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to some embodiments, as shown in FIG. 2, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present disclosure may or may not utilize one or more routers 202. One of ordinary skill in the art would appreciate that there are numerous ways server 203 may connect to WAN 201 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 203 via WAN 201 or other network in numerous ways. For instance, a component or module may connect to the system i) through a computing device 212 directly connected to the WAN 201, ii) through a computing device 205, 206 connected to the WAN 201 through a routing device 204, or iii) through a computing device 208, 210 connected to a wireless access point 207. One of ordinary skill in the art will appreciate that there are numerous ways that a component or module may connect to server 203 via WAN 201 or other network, and embodiments of the present disclosure are contemplated for use with any method for connecting to server 203 via WAN 201 or other network. Furthermore, server 203 could be comprised of a personal computing device, such as a smartphone, acting as a host for other computing devices to connect to. In some embodiments, the computing device 205, 206, 208, 210, 212 is the same as or similar to the computing device 100.

The communications means of the system may be any circuitry or other means for communicating data over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

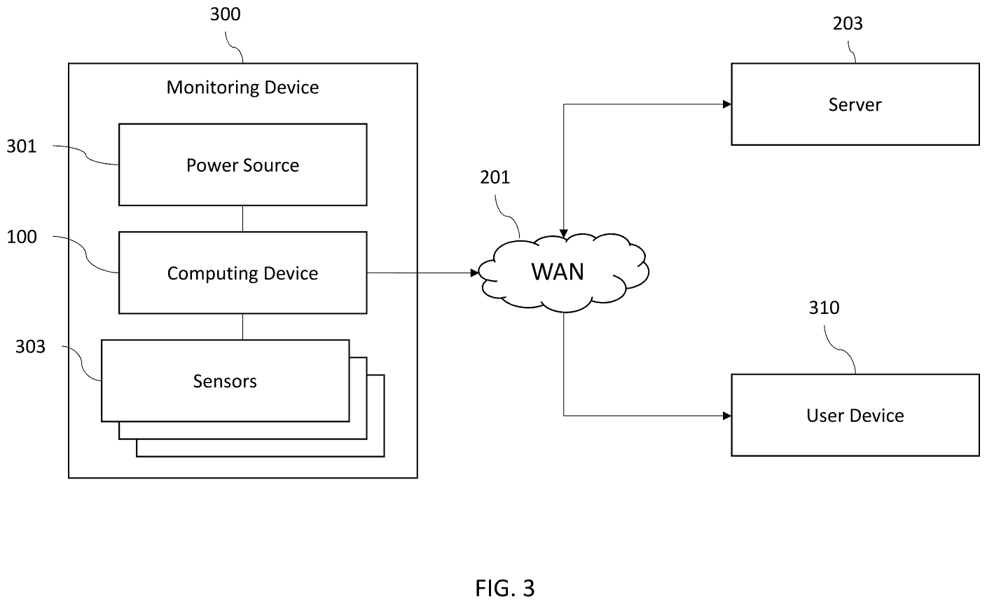

Referring now to FIG. 3 shown is a block diagram of a water quality monitoring system according to some embodiments of the present disclosure. The water quality monitoring system includes a monitoring device 300, capable of real-time monitoring and recording of multiple water quality parameters, including pH, alkalinity, minerals, and other key indicators of water quality. The monitoring device 300 is installed in a water tank, for example a toilet reservoir, to monitor the water quality for an end user, for example at a residential or commercial location. In some embodiments, installing the monitoring device 300 in a toilet reservoir may be desirable as the water in a toilet reservoir is regularly changed out. Regular change out of the water being monitored enables the monitoring device 300 to track water quality parameters in real time, or close to real time, providing more accurate measurements of the current water quality. Additionally, regular change out of the water allows for solutions used to clean or calibrate the monitoring system to be flushed out and prevent false readings.

The monitoring device 300 includes a computing device 100 connected to a power source 301 and one or more sensors 303. The power source 301 is designed to provide electrical power to the one or more sensors 303 and the computing device 100 to ensure data is collected, processed, and transmitted by the one or more sensors 303 and the computing device 100. The power source 301 may be a disposable or rechargeable battery or a wired connection to another power source, for example a wall outlet.

The one or more sensors 303 may include sensors for monitoring the following water quality parameters: pH, alkalinity, mineral levels, hardness, chlorine, TDS, temperature, conductivity, dissolved oxygen, turbidity, and total coliform and E. coli bacteria, although not limited thereto. In some embodiments, the specific water quality parameters being monitored may depend on geographical location and water quality parameters likely to be affected in the geographical region. In some embodiments, the one or more sensors 303 includes a general purpose spectrophotometry type probe that is capable of recording pH, alkalinity and mineral levels of the water. In some embodiments, the one or more sensors 303 are modular and may be added, removed, or switched out as necessary to enable the monitoring device 300 to monitor the desired water quality parameters. In some embodiments, the one or more sensors 303 are probe based, such that the sensors do not rely on chemicals to monitor the given water quality metrics. In this way, the sensors will have a longer lifespan compared to chemical based monitoring sensors which deplete faster compared to probe based sensors. It will be appreciated that many chemical based monitoring systems rely on optics, i.e., viewing a change in color due to a chemical reaction (for example pH strips), and are not suitable for use in a dark environment such as a toilet reservoir.

The one or more sensors 303 transmit various signals indicative of the monitored water quality parameters to the computer device 100 to be processed into readable and displayable data and information. The computing device 100 may relay the information wirelessly to a user device 310 via a WAN 201 or another network based connection. The user device 310 may be a personal computer or smart phone and display the water quality parameter information in real time, show historical trends, display recommendations based on the historical trends and real time data, and display warnings based on the historical trends and real time data.

In some embodiments, the water quality data is transmitted to a server 203. The server 203 may receive water quality data from a plurality of monitoring devices 300 and map the water quality data for a geographical location, for example within a residence, a neighborhood, a city, a county, etc. to show different water quality levels in different areas. This information can be used to track historic and geographical trends in water quality. The historic and geographic trend data may be used to identify if a particular monitoring device 300 is making inaccurate measurements by comparing the measurements to what would be expected in the area and may help identify where a source of contaminates is originating from. For example, if households in an area are detecting high levels of turbidity, this may indicated that a pipe leading to that area is cracked and allowing additional sediment into the water stream.

In some embodiments, the server 203 may push a warning or recommendation to the user device 310 based on the water quality data received from the plurality of monitoring devices. For example, if a monitoring device 300 in a particular household does not detect abnormal levels of sediment but monitoring devices 300 in the neighborhood are detecting high levels of sediment, the server 203 can send a notification, for example a text message, to the user device 310 indicating an increased likelihood that there may be sediment in the water. In some embodiments, a comparison of the current and historic data from various monitoring device 300 in the area can be retrieved and displayed on the user device 310. In some embodiments, the geographic and historic water quality data may be used by regulatory agencies, for example the World Health Organization (WHO) or the Environmental Protection Agency (EPA), to guide and establish water quality guidelines. In some embodiments, the historic trend data may be used to determine when the one or more sensors 303 should be recalibrated. For example, the trend data may show less accurate measurements compared to previous readings or relatively compared to nearby monitoring devices, thereby indicating the sensors 303 may need recalibration. In some embodiments, the one or more sensors 303 may be calibrated to perform a certain number of measurements or for a particular length of time before being recalibrated.

In some embodiments, the water quality data from the monitoring device 300 can be used to generate additional insights and warnings over time for the consumer, utility or water provider. For example, the monitoring device 300 may directly measure data relating to the presence of certain minerals in the water and advise the consumer they may need to consider a modification to or addition of a softening system to remove the detected minerals. In some embodiments, the monitoring device 300 may not directly measure lead; however, the monitoring device 300 may use measurements of the pH, alkalinity, and other water quality parameters to calculate the Langelier Saturation Index (LSI), indicating there is an increased danger of lead leaching out of the pipe. In some embodiments, the calculations and determinations of inferred metrics is carried out by the computing device 100 and transmitted directly to the user device 310. In some embodiments, the calculations and determinations of inferred metrics is carried out by the server 203 based on data from a plurality of monitoring devices 300 in the area and transmitted to the user device 310.

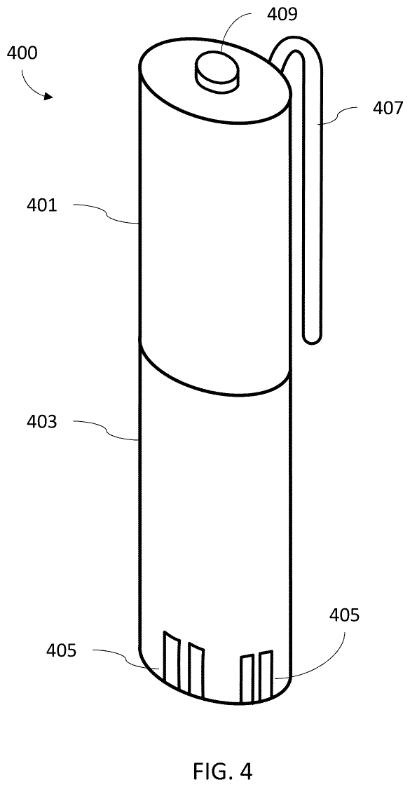

Referring now to FIG. 4, shown is water quality monitoring device 400 according to some embodiments of the present disclosure. The water quality monitoring device 400 may be the same as or similar to the monitoring device 300. The water quality monitoring device 400 includes a housing having an upper chamber 401 and a lower chamber 403. The upper chamber 401 being water tight or semi-water tight and the lower chamber 403 having slots 405 to allow water into the lower chamber 403. The monitoring device 400 also includes a hook 407, or other attachment means, connected to the upper chamber 401 for installing the monitoring device 400 on a water tank or reservoir. When the monitoring device 400 is installed the lower chamber 403 will be at least partially submerged in water.

Similar to the monitoring device 300, the monitoring device 400 may include a computing device 100 and a power source 301 in the upper chamber 401. In this way, the electronics, i.e., the computing device 100 and the power source 301 are housed in the water tight or semi water tight upper chamber 401. The monitoring device 400 also includes one or more sensors 303 which are at least partially disposed in the lower chamber 403 and in data communication with the computing device 100. When the monitoring device 400 is installed, the one or more sensors 303 will be sufficiently submerged in water to be able to take measurements of the water quality parameters of the given sensor.

In some embodiments, the monitoring device 400 includes a tab 409 attached to a top of the upper chamber 401 to facilitate removal of the top of the upper chamber 401. Removing the top of the upper chamber 401 may provide access to the computing device 100 and the power source 301. In this way, the power source 301 can be replaced or recharged. In some embodiments, the lower chamber 403 is removable from the upper chamber 401, thereby exposing the one or more sensors 303 disposed therein. In this way, the one or more sensors 303 may be added, removed or replaced as necessary to monitor the desired water quality metrics.

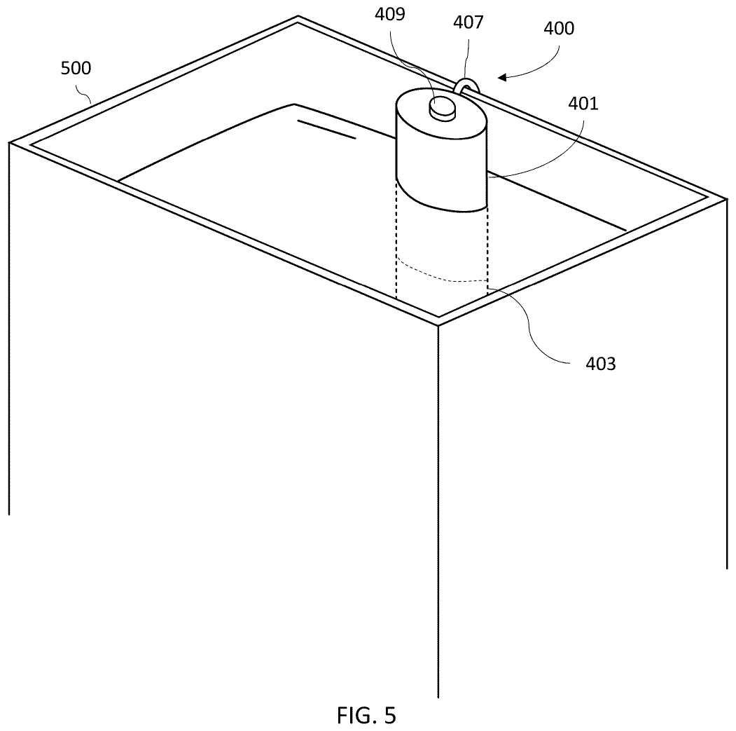

Referring now to FIG. 5, shown is the water quality monitoring device 400 installed in a water tank 500 according to some embodiments of the present disclosure. When the monitoring device 400 is installed in the water tank 500, the hook 407 latches over a side wall of the water tank 500 to secure and suspend the monitoring device in water. In some embodiments, the hook 407 may be flat or substantially flat, for example being a piece of sheet metal, such that a cover may be placed over the tank 500. As illustrated in FIG. 5, the upper chamber 401 is partially submerged and the lower chamber 403 is fully submerged in the water in the water tank 500; however, it is understood that the upper chamber 401 may be partially submerged, fully submerged, or not submerged and the lower chamber 403 may be partially or fully submerged provided the one or more sensors 303 are able to monitor the given water quality metrics. In some embodiments, the water tank 500 is a toilet reservoir and the water therein is regularly changed out, for example, every time the toilet is used.

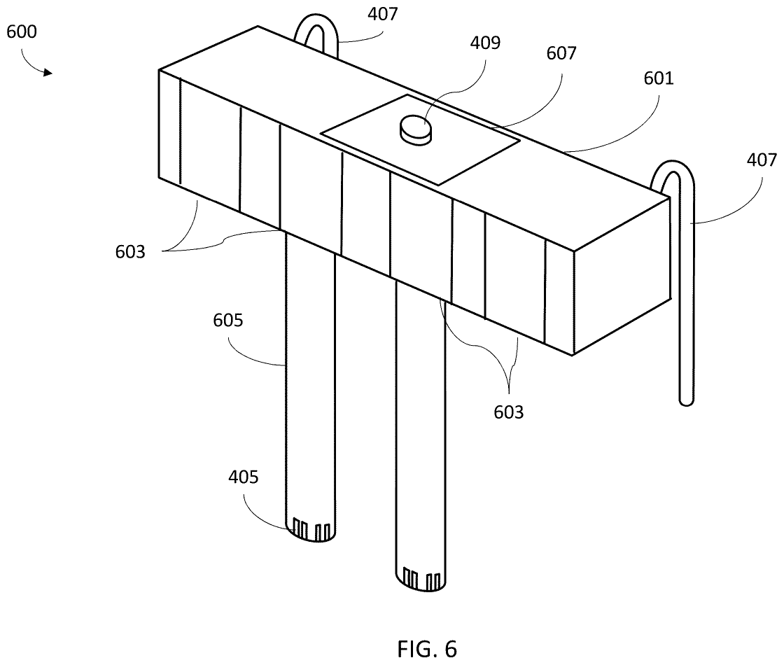

Referring now to FIG. 6, shown is modular water quality monitoring device 600 according to some embodiments of the present disclosure. the modular water quality monitoring device 600 includes a hub 601. The hub 601 may be similar to the upper chamber 401 and may be water tight or semi water tight. A computing device 100 and a power source 301 are housed in the hub 601 and the computing device 100 is electronically connected to the plurality of bays 603. The bays 603 function as a port for receiving a sensor housing 605. In this way, sensors housing 605, having one or more sensors 303 therein, are connected and disconnected from the hub 601 allowing for modular swapping of different sensors to monitor the desired water quality metrics. In some embodiments, the bays 603 and the sensor housings 605 are coupled together forming a mechanical connection between the sensor housing 605 and the hub 601 and an electrical connection between the computing device 100 and the one or more sensors. Although FIG. 6 depicts four bays 603, there may be as many or as few bays 603 as desired for a particular application to support additional sensor housings 605 and sensors.

The hub 601 is installed in water tank via one or more hooks 407 in a similar manner to the monitoring device 400. When installed, the hub 601 may be suspended over, partially or fully submerged in the water in the water tank, provided that when the sensor housings 605 are attached to the hub 601, the sensor housing are at least partially submerged in the water to allow for the one or more sensors 303 in the sensors housing 605 to take measurements of the given water quality parameters being monitored. In some embodiments, each of the sensor housings 605 house a different sensor or group of sensors from other sensors housings 605 connected to the hub 601. In some embodiments, the sensor housings 605 house duplicate sensors to take redundant measurements. In such embodiments, the redundant measurements may be used to check if the sensors are calibrated correctly.

In some embodiments, the modular monitoring device 600 includes a tab 409 attached to a lid 607 of the hub 601 to facilitate removal of the lid 607. Removing the lid 607 may provide access to the computing device 100 and the power source 301 housed in the hub 601. In this way, the power source 301 can be replaced or recharged.

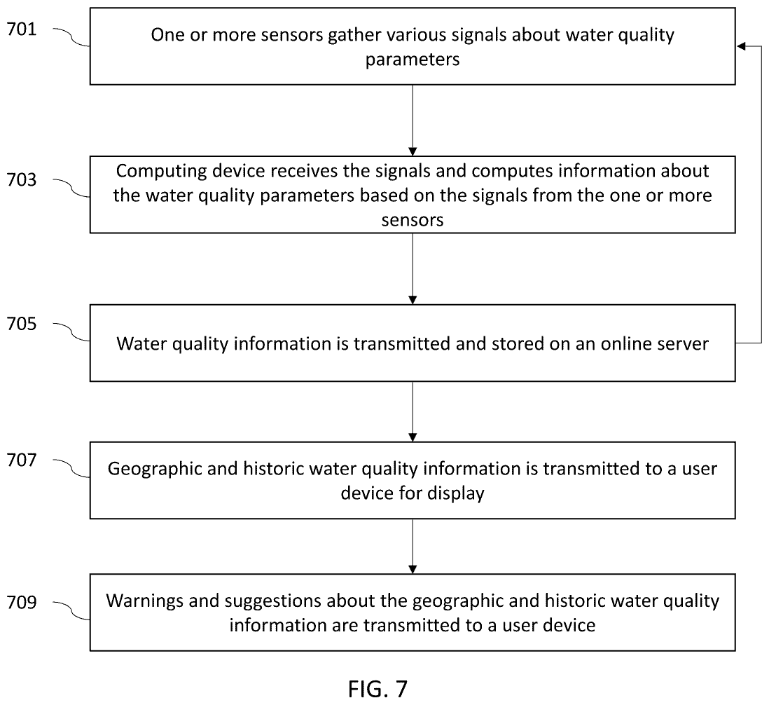

Referring now to FIG. 7, shown is a flow diagram depicting a method of using a water quality monitoring device according to some embodiments of the present disclosure. once the water quality monitoring device is installed in a body of water, at step 701 the one or more sensors 303 of the water quality monitoring system gather signals about given water quality parameters being monitored. The one or more sensors 303 may take continuous readings of the water quality parameters or may take readings at set intervals, for example when the water being monitored is changed out. At step 703, the signals about the given water quality parameters being monitored are received by a computing device 100 and the information/data about the water quality parameters being monitored is computed from the signals received from the one or more sensors 303. At step 705, the computed information about the water quality parameters is transmitted and stored on a server 203.

In some embodiments, step 701, step 703, and step 705 are repeated, gathering additional signals and information about the water quality parameters. In some embodiments, when step 701, step 703, and step 705 are repeated, the same water quality monitoring device and one or more sensors 303 gather the additional signals and information about the water quality parameters, mapping historic trends over time of the water quality parameters. In some embodiments, when step 701, step 703, and step 705 are repeated, different water quality monitoring device and one or more sensors 303 at different geographic locations gather the additional signals and information about the water quality parameters, mapping trends of the water quality parameters in a geographic location.

At step 707, geographic and/or historic trends in the water quality parameters compiled on the server 203 are transmitted to a user device 310 for display. At step 709, warning and suggestions about the geographic and historic water quality information is transmitted to a user device 310, for example the presence of minerals in the water may prompt the suggestion that a softener be added to the user's water system or issuing a turbidity warning for a geographical area based on nearby water quality monitoring systems detecting high turbidity. In some embodiments, machine learning or artificial intelligence may be used to process the geographic and historic water quality information to detect trends and predict/infer additional water quality parameters. For example, in embodiments where the water quality monitoring system is not monitoring heavy metals, based on historic data high levels of turbidity may indicate a heightened chance of heavy metals being in the water.

In some embodiments, the measurement of the various water quality parameters are compared against a threshold value and if the measured water quality parameter meets or exceeds the threshold value the server 203 or the computing device 100 may send a notification or warning to the user device that the particular water quality parameter met or exceeded the threshold value. In some embodiments, the threshold value for each water quality parameter is set manually by the user. In some embodiments, the threshold value for each water quality parameter is determined by regulatory agencies, for example the EPA or WHO, or service/utility providers. In some embodiments, the threshold value for each water quality parameter is determined by machine learning or artificial intelligence.

The water quality monitoring devices disclosed herein may be installed in a variety of environments for multiple different use cases. For example, fire hydrant monitoring: the water quality monitoring device may be installed on fire hydrants, this embodiment monitors water quality across a city or municipality. It helps detect contamination or pollutants in the public water supply, ensuring safety for large populations. Data can be shared with city officials for real-time analysis and response.

Subdivision/Apartment Building Connection: the water quality monitoring device may be positioned at the main water connection to a subdivision or apartment complex, this device provides collective water quality data to the property owners association or building management. It ensures that all residents receive safe and clean water and can detect issues affecting multiple households.

Inside Smart Appliances: the water quality monitoring device may be integrated into smart appliances like refrigerators, dishwashers, and washing machines, the device monitors water quality used in these appliances. This ensures that water for drinking, cleaning, and washing is safe, and can alert users to potential issues directly through the appliance interface or connected apps.

Home Water Filtration Systems: the water quality monitoring device may be installed in conjunction with home water filtration or softening systems, the device monitors the effectiveness of the filtration process. It provides real-time feedback on water quality before and after filtration, ensuring the system is functioning correctly and alerting users to replace filters as needed.

Hotel/Resort Water Systems: the water quality monitoring device may be deployed within hotel or resort water systems, this embodiment ensures guests have access to high-quality water throughout their stay. It monitors the water supplied to rooms, pools, and spas, providing management with data to maintain health standards and improve guest satisfaction.

Cruise Ship Water Systems: the water quality monitoring device may be installed in the water supply systems of cruise ships, the device ensures passengers and crew have access to safe drinking water. It monitors water quality across different areas of the ship, providing real-time data to the ship's engineering team to address any issues promptly.

Commercial Building Water Systems: the water quality monitoring device may be positioned within commercial buildings such as offices or shopping malls, the device monitors water quality supplied to restrooms, kitchens, and drinking fountains. This ensures the safety and well-being of employees and visitors and aids in maintaining regulatory compliance.

Agricultural Irrigation Systems: the water quality monitoring device may be used within agricultural settings, the device monitors the quality of water used for irrigation. This helps farmers ensure that crops receive clean water, free of harmful contaminants, which can affect crop health and yield.

School/University Campus Water Systems: the water quality monitoring device may be installed across school or university campuses, the device ensures safe drinking water for students and staff. It can monitor water quality in cafeterias, dormitories, and gymnasiums, providing data to facility managers for prompt action.

Sports Arenas and Stadiums: the water quality monitoring device may be positioned in the water systems of sports arenas and stadiums, the device ensures high-quality water for athletes, staff, and spectators. It monitors water used in restrooms, concession stands, and locker rooms, maintaining health standards during events.

Public Parks and Recreational Areas: the water quality monitoring device may be installed in public parks and recreational areas, this embodiment monitors water quality in fountains, splash pads, and drinking stations. It provides data to city maintenance teams, ensuring safe and clean water for public use.

Marinas and Boating Facilities: the water quality monitoring device may be used in marinas and boating facilities, the device monitors the quality of water supplied to boats and dockside facilities. It ensures that boaters have access to safe water for drinking and other uses, while also monitoring the environmental impact on local water bodies.

Emergency Relief Water Systems: the water quality monitoring device may be deployed in emergency relief situations, such as natural disasters, this portable device monitors the quality of water supplied to temporary shelters and relief centers. It helps ensure that affected populations receive safe drinking water during crises.

Industrial Facility Water Systems: the water quality monitoring device may be installed in industrial facilities, the device monitors water quality used in manufacturing processes. It helps ensure that water meets specific standards required for production, protecting equipment and ensuring product quality.

Recreational Vehicles (RVs): the water quality monitoring device may be integrated into the water systems of recreational vehicles, the device monitors the quality of water used for drinking, cooking, and bathing. It provides real-time data to RV owners, ensuring safe water during travel.

Residential Garden Irrigation Systems: the water quality monitoring device may be installed in home garden irrigation systems, this embodiment monitors the quality of water used for gardening. It ensures that plants receive clean water, free of harmful contaminants, promoting healthier growth.

Public Transportation Hubs: the water quality monitoring device may be positioned in public transportation hubs such as airports, bus terminals, and train stations, the device monitors water quality in restrooms and drinking fountains, ensuring safe water for travelers and staff.

Aquariums and Zoos: the water quality monitoring device may be used in aquariums and zoos, the device monitors the quality of water in exhibits and enclosures, ensuring the health and well-being of aquatic and terrestrial animals.

Community Centers and Gyms: the water quality monitoring device may be Installed in community centers and gyms, the device monitors water quality in drinking fountains, showers, and pools, ensuring a safe and healthy environment for members and visitors.

Camping and Outdoor Recreation Areas: the water quality monitoring device may be portable and easy to install, the device can be used in camping and outdoor recreation areas to monitor the quality of water from natural sources or campsite facilities, ensuring safe water for campers and hikers.

Although the invention has been explained in relation to some embodiments, it is to be understood that many other possible modifications and variations can be made without departing from the spirit and scope of the invention.

The various aspects illustrated by logical blocks, modules, circuits, processes, algorithms, and algorithm steps described above may be implemented as electronic hardware, software, or combinations of both. Certain disclosed components, blocks, modules, circuits, and steps are described in terms of their functionality, illustrating the interchangeability of their implementation in electronic hardware or software. The implementation of such functionality varies among different applications given varying system architectures and design constraints. Although such implementations may vary from application to application, they do not constitute a departure from the scope of this disclosure.

Aspects of embodiments implemented in software may be implemented in program code, application software, application programming interfaces (APIs), firmware, middleware, microcode, hardware description languages (HDLs), or any combination thereof. A code segment or machine-executable instruction may represent a procedure, a function, a subprogram, a routine, a subroutine, a module, a software package, a class, or any combination of instructions, data structures, or program statements. A code segment may be coupled to, or integrated with, another code segment or an electronic hardware by passing or receiving information, data, arguments, parameters, memory contents, or memory locations. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the disclosed functions may be embodied, or stored, as one or more instructions or code on or in memory. In the embodiments described herein, memory includes non-transitory computer-readable media, which may include, but is not limited to, media such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROM, DVD, and any other digital source such as a network, a server, cloud system, or the Internet, as well as yet to be developed digital means, with the sole exception being a transitory propagating signal. The methods described herein may be embodied as executable instructions, e.g., “software” and “firmware,” in a non-transitory computer-readable medium. As used herein, the terms “software” and “firmware” are interchangeable and include any computer program stored in memory for execution by personal computers, workstations, clients, and servers. Such instructions, when executed by a processor, configure the processor to perform at least a portion of the disclosed methods.

As used herein, an element or step recited in the singular and proceeded with the word “a” or “an” should be understood as not excluding plural elements or steps unless such exclusion is explicitly recited. Furthermore, references to “one embodiment” of the disclosure or an “exemplary” or “example” embodiment are not intended to be interpreted as excluding the existence of additional embodiments that also incorporate the recited features. Likewise, limitations associated with “one embodiment” or “an embodiment” should not be interpreted as limiting to all embodiments unless explicitly recited.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

The disclosed systems and methods are not limited to the specific embodiments described herein. Rather, components of the systems or steps of the methods may be utilized independently and separately from other described components or steps.

This written description uses examples to disclose various embodiments, which include the best mode, to enable any person skilled in the art to practice those embodiments, including making and using any devices or systems and performing any incorporated methods. The patentable scope is defined by the claims and may include other examples that occur to those skilled in the art. Such other examples are intended to be within the scope of the claims if they have structural elements that do not differ from the literal language of the claims, or if they include equivalent structural elements with insubstantial differences form the literal language of the claims.

What is claimed is:

1. A device installed in a toilet reservoir for monitoring water quality, the device comprising:
a housing configured to be at least partially submerged in water in the toilet reservoir;
a computing device disposed in the housing and in data communication with a database; and
at least one sensor in data communication with the computing device, the at least one sensor measuring at least one water quality parameter;
wherein the at least one sensor takes a measurement of the at least one water quality parameter of the water in the toilet reservoir and transmits the measurement to the computing device and the computing device stores the measurement on the database.

2. The device of claim 1, wherein when water in the toilet reservoir is changed out the at least one sensor takes a second measurement of the at least one water quality parameter and transmits the second measurement to the computing device and the computing device stores the second measurement on the database.

3. The device of claim 2, wherein each time water in the toilet reservoir is changed out the at least one sensor takes a subsequent measurement of the at least one water quality parameter and transmits the subsequent measurement to the computing device and the computing device stores the subsequent measurement on the database.

4. The device of claim 3, wherein the computing device determines a second water quality parameter based on the measurement, the second measurement, and the subsequent measurement of the at least one water quality parameter stored on the database.

5. The device of claim 4, wherein the measurement of the at least one water quality parameter, the second measurement of the at least one water quality parameter, the subsequent measurement of the at least one water quality parameter, and the second water quality parameter are transmitted and displayed on a user device.

6. The device of claim 1, wherein the computer device compares the measurement of the at least one water quality parameter against a threshold value and when the measurement of the at least one water quality parameter meets or exceeds the threshold value, the computing device transmits an alert to a user device.

7. The device of claim 1, wherein the at least one sensor is a plurality of sensors.

8. The device of claim 1, wherein the at least one sensor is a multi-parameter probe.

9. The device of claim 1, wherein the at least one water quality parameter is one or more of pH, alkalinity, mineral levels, hardness, chlorine, TDS, temperature, conductivity, dissolved oxygen, turbidity, and total coliform and E. coli bacteria.

10. A system for monitoring water quality, the system comprising:
a server;
a plurality of monitoring devices installed in a respective water tank, each monitoring device comprising:
a housing configured to be at least partially submerged in water in the respective water tank;
a computing device disposed in the housing and in data communication with the server; and
at least one sensor in data communication with the computing device, the at least one sensor measuring at least one water quality parameter;
wherein the at least one sensor takes a measurement of the at least one water quality parameter in the respective water tank and transmits the measurement to the computing device and the computing device stores the measurement on the server;
wherein the server compiles and transmits the measurement from each of the monitoring device to a user device to be displayed.

11. The system of claim 10, wherein when water in the respective water tank is changed out the respective at least one sensor of the respective monitoring device takes a second measurement of the at least one water quality parameter and transmits the second measurement to the respective computing device and the respective computing device stores the second measurement on the server.

12. The system of claim 11, wherein each time water in the respective water tank is changed out the respective at least one sensor takes a subsequent measurement of the at least one water quality parameter and transmits the subsequent measurement to the respective computing device and the respective computing device stores the subsequent measurement on the server.

13. The system of claim 12, wherein the server determines a second water quality parameter based on the respective measurement of the at least one water quality parameter, the respective second measurement of the at least one water quality parameter, and the respective subsequent measurement of the at least one water quality parameter of each of the monitoring devices stored on the server.

14. The system of claim 13, wherein the respective measurement of the at least one water quality parameter, the respective second measurement of the at least one water quality parameter, the respective subsequent measurement of the at least one water quality parameter, and the second water quality parameter are transmitted and displayed on a user device.

15. A method for using a device installed in a toilet reservoir for monitoring water quality, the method comprising:
measuring at least one water quality parameter using one or more sensors;
transmitting and storing the measurements of the at least one water quality parameter on a server;
determining a second water quality parameter from the measurements of the at least one water quality parameter;
transmitting the measurements of the at least one water quality parameter and the second water quality parameter to a user device for display.

16. The method of claim 15, further comprising comparing the measurements of the at least one water quality parameter against a first threshold value; and
transmitting a first alert to the user device when the measurements of the at least one water quality parameter meets or exceeds the first threshold value.

17. The method of claim 16, further comprising comparing the second water quality parameter against a second threshold value; and
transmitting a second alert to the user device when the second water quality parameter meets or exceeds the second threshold value.
